(19) United States
(12) Patent Application Publication
(10) Pub. No.: US 2023/0270905 A1
(43) Pub. Date: Aug. 31, 2023

(54) ROBOT

(71) Applicant: SAMSUNG ELECTRONICS CO., LTD., Suwon-si (KR)

(72) Inventors: Takeshi YAMAGISHI, Yokohama-shi (JP); Hongkue LEE, Yokohama-shi (JP); Yuki ABE, Yokohama-shi (JP)

(73) Assignee: SAMSUNG ELECTRONICS CO., LTD., Suwon-si (KR)

(21) Appl. No.: 18/143,279

(22) Filed: May 4, 2023

Related U.S. Application Data
(63) Continuation of application No. PCT/KR2021/019379, filed on Dec. 20, 2021.

(30) Foreign Application Priority Data
Dec. 24, 2020 (JP) 2020-215047

(51) Int. Cl. A61L 2/24; B25J 5/00; B25J 11/00; A61L 2/10; A61L 2/22; A61L 2/18; A61L 9/20; A61L 9/14; A61L 9/04; A61L 9/12

(52) U.S. Cl. CPC A61L 2/24; B25J 5/00; B25J 11/0085; A61L 2/10; A61L 2/22; A61L 2/183; A61L 9/20; A61L 9/14; A61L 9/046; A61L 9/12; A61L 2202/11; A61L 2202/15; A61L 2202/122; A61L 2202/16; A61L 2209/12; A61L 2209/134

(57) ABSTRACT

The disclosed robot enables the efficient transportation of objects scattered over a wide area. The disclosed robot may include: a main body having one or more support; a moving mechanism for moving the main body; and a manipulator installed on one support, among the one or more support, thereby having the one support as a rotating shaft so as to be rotatable by 360° or higher

### CROSS-REFERENCE TO RELATED APPLICATION(S)

This application is a Bypass Continuation Application of PCT International Application No. PCT/KR2021/019379, filed on Dec. 20, 2021, which is based on and claims priority to Japanese Patent Application No. 2020-215047, filed on Dec. 24, 2020, in the Japanese Intellectual Property Office, the disclosure of which are incorporated by reference herein in their entireties

### BACKGROUND

### 1. Field

The disclosure relates to a robot.

### 2. Description of Related Art

In patent document 1 (Japanese Unexamined Patent Application No. 2007-90493), an arm mounted robot is disclosed, wherein a robot main body has a storage space for accommodating an entire arm.

In patent document 2 (Japanese Unexamined Patent Application No. 2018-535163), a system including an automated guided vehicle (AGV), a loading table which is disposed on the AGV and of which a structure and size are determined to support two or more storage containers, a frame extending from the AGV, and a robot arm installed on the frame is disclosed.

In patent document 3 (Japanese Unexamined Patent Application No. 22-128973), a moving object is disclosed, wherein an arm is a multi joint robot arm, a root part is connected to an upper end portion of a compartment, and when a table is rotated with respect to a frame by the root part, the arm is also rotated to follow the table, and thus a central position of the moving object is moved in a turning direction of the arm.

### SUMMARY

Provided is a robot including a manipulator having only a relatively small operating range cannot effectively transport objects distributed in a wide area.

The present disclosure is directed to efficiently transporting objects distributed in a wide area.

One aspect of the present disclosure provides a robot including a main body including at least one support column, a moving mechanism configured to move the main body, and a manipulator installed on one support column of the at least one support column which can be rotated 360° or more about a rotation axis of the one support column.

The at least one support column may include a support column in a horizontal direction, and the one support column may be the support column in the horizontal direction.

In this case, the at least one support column may further include a first support column in a vertical direction, and a first end portion of the support column in the horizontal direction may be coupled to an upper end portion of the first support column in the vertical direction. In addition, the at least one support column may further include a second support column in the vertical direction, and a second end portion of the support column in the horizontal direction may be coupled to an upper end portion of the second support column in the vertical direction.

Alternatively, the at least one support column may further include a first support column in a vertical direction, and a first end portion of the support column in the horizontal direction may be installed on the first support column to be moved in the vertical direction. In addition, the at least one support column may further include a second support column in the vertical direction, and a second end portion of the support column in the horizontal direction may be installed on the second support column to be moved in the vertical direction.

The at least one support column may include a support column in a vertical direction, and the one support column may be the support column in the vertical direction.

The robot may further include a measurement mechanism configured to measure a three-dimensional shape of an object.

The robot may further include a treatment mechanism configured to perform at least one treatment among sterilization, bacteria elimination, disinfection, drying, and deodorization treatments on an object.

The main body may further include a holder rotated within a horizontal plane to hold an object.

In this case, the robot may further include a measurement mechanism configured to measure a three-dimensional shape of a corresponding object by rotating the holder where the object is loaded. In addition, the robot may further include a controller configured to control the moving mechanism and the manipulator to relocate the object to a relocation position on the basis of the three-dimensional shape of the object measured by the measurement mechanism and a relationship between the three-dimensional shape and the relocation position of the object.

Alternatively, the robot may further include a treatment mechanism configured to perform at least one treatment among sterilization, bacteria elimination, disinfection, drying, removal, and deodorization treatments on the object by rotating the holder where the object is loaded. In addition, the treatment mechanism may perform the at least one treatment on the object moved at a specific position by rotating the holder.

A cleaner unit may be installed on the manipulator. The cleaner unit may further include a treatment mechanism configured to perform at least one treatment among sterilization, bacteria elimination, disinfection, drying, and deodorization treatments on the object.

In addition, another aspect of the present disclosure provides a robot including a main body having a first support column in a vertical direction and a support column of which a first end portion is coupled to an upper end portion of the first support column in the vertical direction and which is formed in a horizontal direction, a moving mechanism configured to move the main body, and a manipulator installed on the support column in the horizontal direction.

The main body may further include a second support column in the vertical direction, and a second end portion of the support column in the horizontal direction may be coupled to an upper end portion of the second support column in the vertical direction.

In addition, still another aspect of the present disclosure provides a robot including a main body having a first support column in a vertical direction, a second support column in the vertical direction, and a support column of which a first end portion is installed on the first support column to be moved in the vertical direction and a second end portion is installed on the second support column to be moved in the vertical direction, a moving mechanism configured to move the main body, and a manipulator installed on the support column in the horizontal direction.

In addition, yet another aspect of the present disclosure provides a robot including a main body having a holder rotated within a horizontal plane to hold an object, a moving mechanism configured to move the main body, and a manipulator installed at a position tangent to an outer circumference of the holder.

According to the present disclosure, objects distributed in a wide area can be transported efficiently.

### BRIEF DESCRIPTION OF THE DRAWINGS

The above and other aspects, and features of certain embodiments of the present disclosure will be more apparent from the following description taken in conjunction with the accompanying drawings, in which:

FIG. 1 is a side view illustrating a robot according to a first embodiment of the present disclosure.

FIG. 2 is a front perspective view illustrating the robot according to the first embodiment of the present disclosure.

FIG. 3 is a front perspective view illustrating the robot according to the first embodiment of the present disclosure.

FIG. 4 is a front perspective view illustrating the robot according to the first embodiment of the present disclosure.

FIG. 5 is a view illustrating a rotation direction of each part of a manipulator of the robot according to the first embodiment of the present disclosure.

FIG. 6 is a schematic view illustrating operation of the robot when a three-dimensional information acquisition device measures a three-dimensional shape of an object according to the first embodiment of the present disclosure.

FIG. 7 is a schematic view illustrating operation of the robot when a treatment device for sterilization and the like performs sterilization, bacteria elimination, disinfection, drying, and deodorization on the object according to the first embodiment of the present disclosure.

FIGS. 8A to 8C are schematic views illustrating operation of the robot when carrying the object into a treatment box for sterilizing and the like and performing sterilization, bacteria elimination, disinfection, drying, and deodorization on the object according to the first embodiment of the present disclosure.

FIG. 9 is a view illustrating a configuration in which a cleaner unit of the robot is installed according to the first embodiment of the present disclosure.

FIG. 10 is a view illustrating an example of a configuration of a control device of the robot according to the first embodiment of the present disclosure.

FIG. 11 is a flowchart illustrating an example of an operation of the control device of the robot according to the first embodiment of the present disclosure.

FIG. 12 is a side view illustrating a robot according to a second embodiment of the present disclosure.

FIG. 13 is a side view illustrating a robot according to a third embodiment of the present disclosure.

FIG. 14 is a top perspective view illustrating a robot according to a fourth embodiment of the present disclosure.

### DETAILED DESCRIPTION

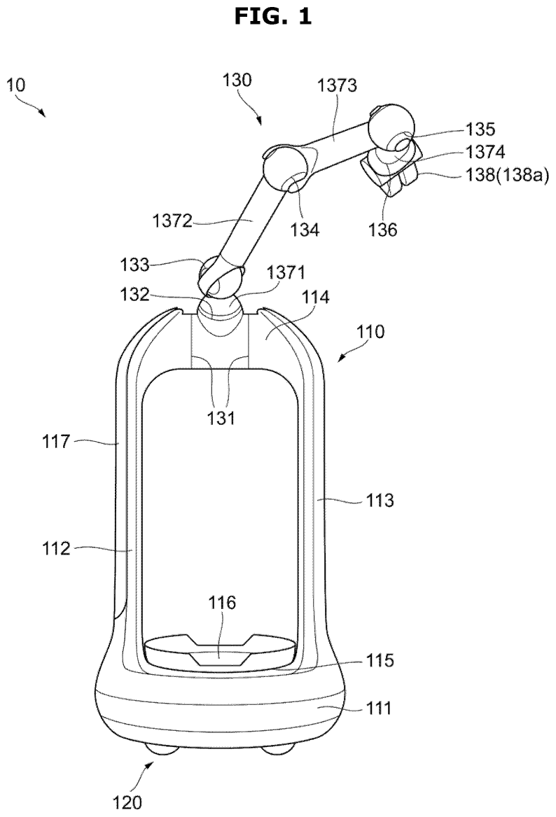

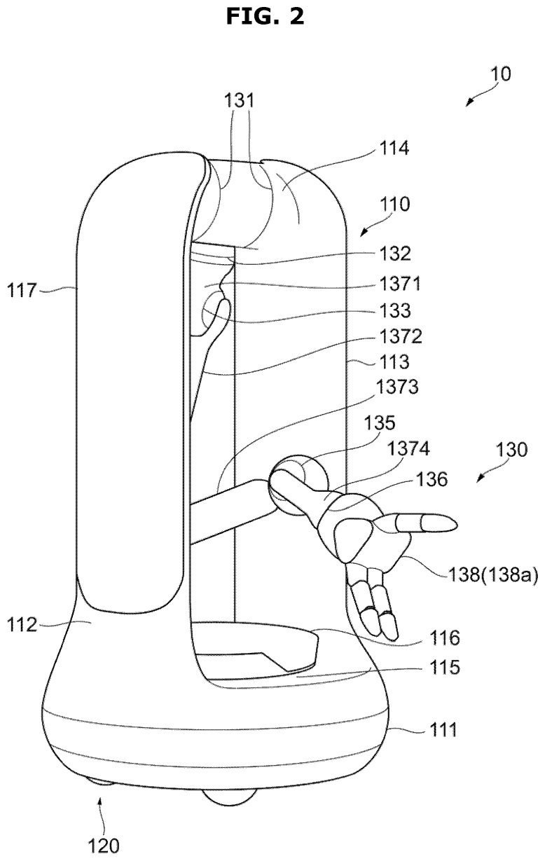

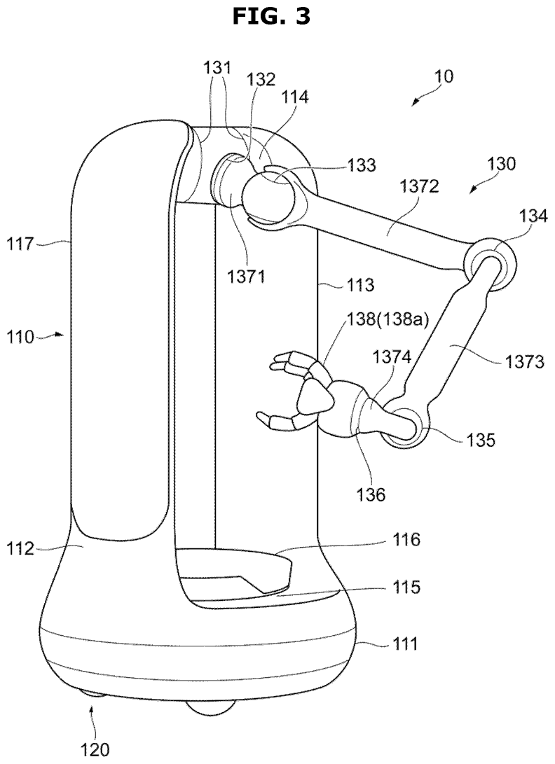

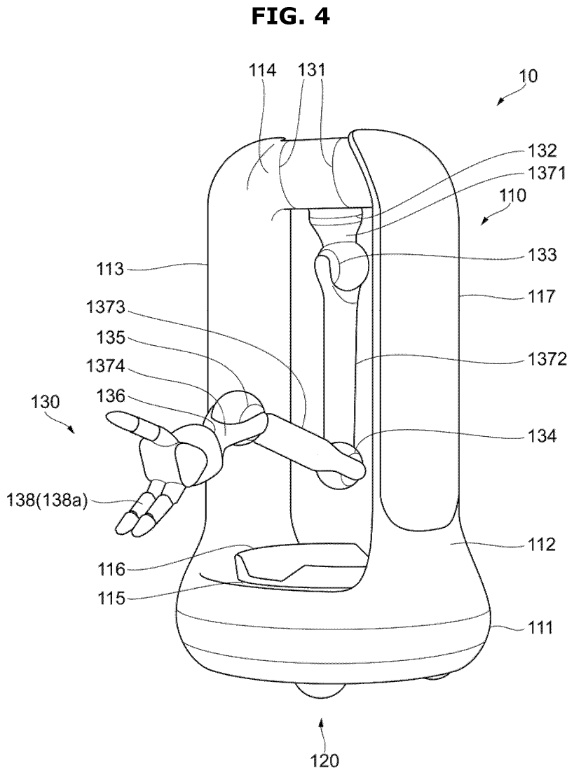

FIG. 1 is a side view illustrating a robot 10 according to a first embodiment of the present disclosure, and FIGS. 2 to 4 are front perspective views illustrating the robot 10 according to the first embodiment of the present disclosure. The robot 10 includes a main body 110, a moving mechanism 120, and a manipulator 130.

The main body 110 has a door shape as illustrated in FIG. 1. That is, the main body 110 includes a base 111, a first vertical support column 112, a second vertical support column 113, and a horizontal support column 114. The base 111 is a base of the main body 110. The first vertical support column 112 is a first support column provided on the base 111 in a vertical direction and is one example of a first support column in the vertical direction. The second vertical support column 113 is a second support column provided on the base 111 in the vertical direction and is one example of a second support column in the vertical direction. The horizontal support column 114 is a support column provided in a horizontal direction to be coupled to an upper end portion of the first vertical support column 112 in the vertical direction and an upper end portion of the second vertical support column 113 in the vertical direction and is one example of a support column in the horizontal direction. In this case, the case of “being vertical” in this specification includes not only a case of matching the vertical direction, but also a case of being inclined with respect to the vertical direction to an extent of achieving an effect of the present embodiment. In addition, the case of “being horizontal” in this specification includes not only a case of matching the horizontal direction, but also a case of being inclined with respect to the horizontal direction to an extent of achieving an effect of the present embodiment.

In addition, the main body 110 includes a rotating table 115 and a transportation table 116 on the base 111. The rotating table 115 is a flat table rotated within a horizontal plane. The transportation table 116 is a dish-shaped table provided on the rotating table 115 to load a transportation target object. In this case, since the transportation table 116 is disposed above the rotating table 115, the transportation table 116 is rotated within a horizontal plane, and since an object loaded on the transportation table 116 is not limited to the transportation target object, the transportation table 116 is one example of a holder rotated within a horizontal plane to hold an object.

In addition, as illustrated in FIGS. 2 to 4, in the main body 110, the first vertical support column 112 includes a display 117 on a front surface of a door shape. The display 117 is used for communication with a user or for providing information to the user.

The moving mechanism 120 is a mechanism for moving the main body 110. The moving mechanism 120 may be implemented by arranging a tire, a crawler, or the like under the main body 110. Specifically, the moving mechanism 120 may allow the robot 10 to move in any direction by arranging three omni wheels under the main body 110.

The manipulator 130 is a part for performing actual operation according to a use of the robot 10. The manipulator 130 is installed on the horizontal support column 114 of the main body 110 and is disposed in the door shape of the main body 110. The manipulator 130 includes a first joint 131, a second joint 132, a third joint 133, a fourth joint 134, a fifth joint 135, a sixth joint 136, a first link 1371, a second link 1372, a third link 1373, a fourth link 1374, and an end effector 138.

The first joint 131 is a part corresponding to a joint installed on the horizontal support column 114 which is a root of the manipulator 130 and is installed to be rotated 360° or more about an axis that corresponds with the horizontal support column 114. Accordingly, the manipulator 130 may lift the object upward as illustrated in FIG. 1 and freely rotate in a left direction and a right direction when viewed from the front as illustrated in FIGS. 2 to 4.

The second joint 132 is a part corresponding to a joint for connecting the horizontal support column 114 and the manipulator 130. The first link 1371 is a part corresponding to a shoulder installed on the second joint 132. The third joint 133 is a part corresponding to a joint of a front end of the first link 1371. The second link 1372 is a part corresponding to an arm installed on the third joint 133. The fourth joint 134 is a part corresponding to a joint of a front end of the second link 1372. The third link 1373 is a part corresponding to an arm installed on the fourth joint 134. The fifth joint 135 is a part corresponding to a joint of a front end of the third link 1373. The fourth link 1374 is a part corresponding to a wrist installed on the fifth joint 135. The sixth joint 136 is a part corresponding to a joint of a front end of the fourth link 1374.

The end effector 138 is a device installed on the sixth joint 136 to perform actual operation according to the use of the robot 10. In this case, a multi-finger hand 138a is illustrated as the end effector 138.

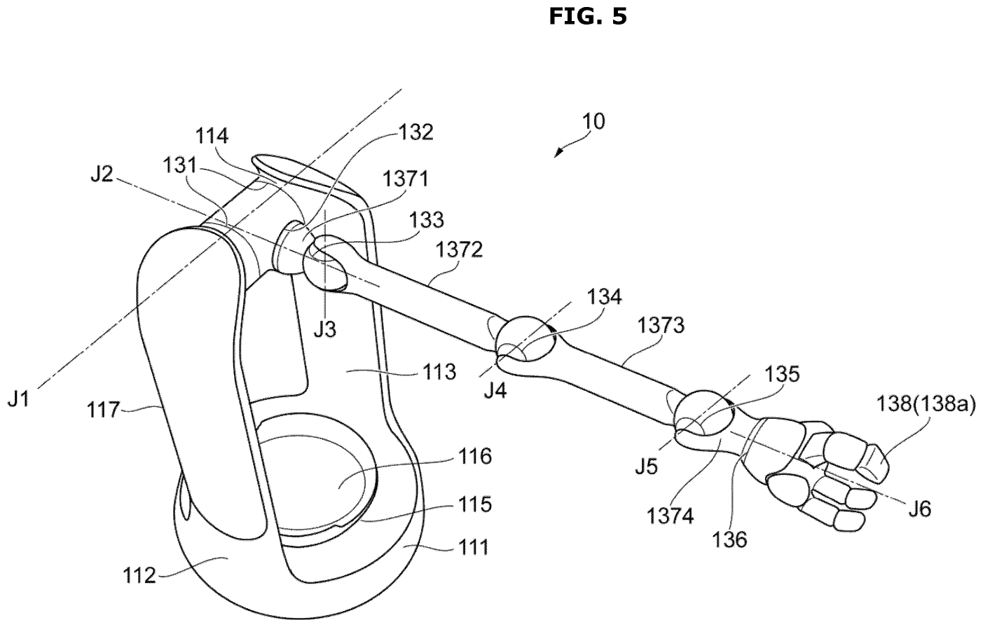

In this case, rotation directions of the joints of the manipulator 130 will be described. FIG. 5 is a view illustrating the rotation directions.

As described above, the first joint 131 rotates about an axis J1, which is a central axis of the horizontal support column 114 of the main body 110, as a rotation axis. The second joint 132 rotates about an axis J2, which is perpendicular to the axis J1, as a rotation axis. The third joint 133 rotates about an axis J3, which is perpendicular to the axis J2, as a rotation axis. The fourth joint 134 rotates about an axis J4, which is perpendicular to the axis J3, as a rotation axis. The fifth joint 135 rotates about an axis J5, which is parallel to the axis J4, as a rotation axis. The sixth joint 136 rotates about an axis J6, which is perpendicular to the axis J5, as a rotation axis. This configuration allows the manipulator to have a wide range of motion.

In addition, in the robot 10 according to the first embodiment, the transportation table 116 on which the object is loaded is rotated so that a three-dimensional information acquisition device disposed on the end effector 138 measures a three-dimensional shape of the object.

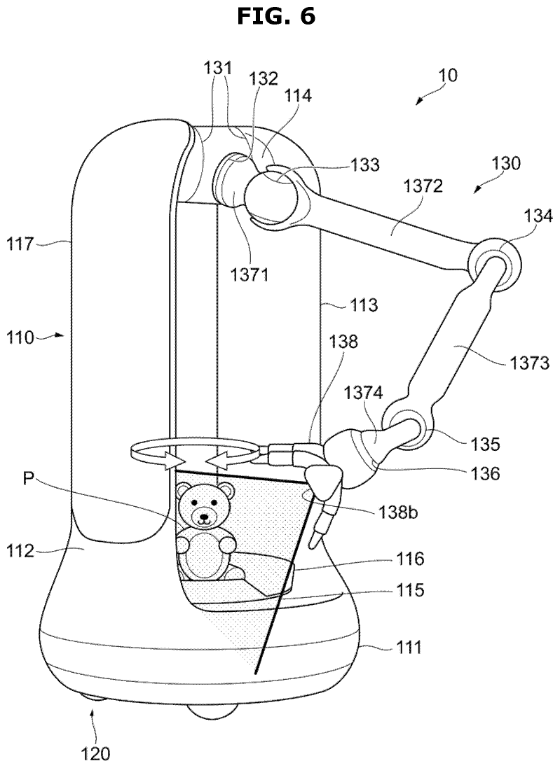

FIG. 6 is a schematic view illustrating operation of the robot 10 for the above description. In the robot 10, a three-dimensional information acquisition device 138b is disposed on the end effector 138. Accordingly, the robot 10 obtains three-dimensional information within a range surrounded by thick lines in a radial direction of a fan shape using the three-dimensional information acquisition device 138b while rotating the transportation table 116 where a puppet P is loaded as indicated by a thick arrow. Accordingly, the three-dimensional information acquisition device 138b may obtain three-dimensional information of the puppet P.

In this case, the three-dimensional information acquisition device 138b may be a camera or a three-dimensional (3D) capture device. In addition, the three-dimensional information acquisition device 138b may also be disposed on the link of the manipulator 130, the first vertical support column 112, a portion beside the transportation table 116 of the second vertical support column 113, or the like instead of the end effector 138. The three-dimensional information acquisition device 138b is one example of a measurement mechanism for measuring a three-dimensional shape of an object by rotating a holder where an object is loaded.

Alternatively, when the three-dimensional information acquisition device 138b is disposed on the manipulator 130, the robot 10 may operate the manipulator 130 to obtain three-dimensional information of the puppet P using the three-dimensional information acquisition device 138b without rotating the transportation table 116. For instance, the manipulator 130 may rotate around puppet P while the information acquisition device 138b is active. In this case, the three-dimensional information acquisition device 138b is one example of a measurement mechanism for measuring a three-dimensional shape of an object.

In addition, the robot 10 according to the first embodiment performs sterilization, bacteria elimination, disinfection, drying, and deodorization of an object using a treatment device for sterilization and the like disposed on the end effector 138 while rotating the transportation table 116 where the object is loaded.

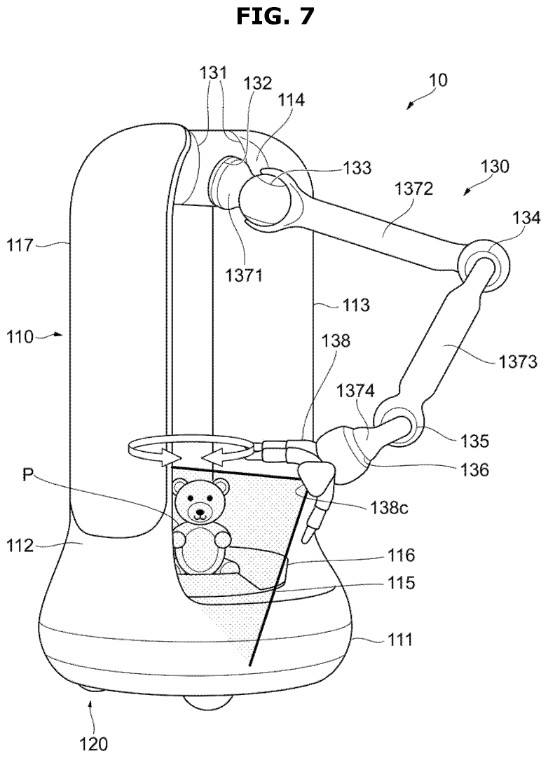

FIG. 7 is a schematic view illustrating operation of the robot 10 for the above description. In the robot 10, a treatment device 138c for sterilization and the like is disposed on the end effector 138. Accordingly, for example, the robot 10 performs sterilization, bacteria elimination, disinfection, drying, and deodorization within a range surrounded by thick lines in a radial direction of a fan shape using the treatment device 138c for sterilization and the like while rotating the transportation table 116 where the puppet P is loaded as indicated by a thick arrow. Accordingly, the treatment device 138c for sterilization and the like performs sterilization, bacteria elimination, drying, and deodorization on the puppet P.

In this case, the treatment device 138c for sterilization and the like may be an ultraviolet (UV) radiator, a bacteria elimination sprayer, an ozone generator, or the like. In addition, the treatment device 138c for sterilization and the like may be a device for performing at least one of sterilization, bacteria elimination, disinfection, drying, and deodorization or a device in which devices each for performing at least one of sterilization, bacteria elimination, disinfection, drying, and deodorization are coupled to perform two or more of sterilization, bacteria elimination, disinfection, drying, and deodorization. In addition, the treatment device 138c for sterilization and the like may also be disposed under the main body 110, such as beside the moving mechanism 120, and on an upper portion of the transportation table 116 of the horizontal support column 114, the first vertical support column 112, a portion beside the transportation table 116 of the second vertical support column 113, or the like instead of the end effector 138. The treatment device 138c for sterilization and the like is one example of a treatment mechanism for performing at least one treatment among sterilization, bacteria elimination, disinfection, drying, and deodorization treatments on an object by rotating a holder where an object is loaded.

Alternatively, when the treatment device 138c for sterilization and the like is disposed on the manipulator 130, the robot 10 may also operate the manipulator 130 to perform sterilization, bacteria elimination, disinfection, drying, and deodorization on the puppet P using the treatment device 138c for sterilization and the like without rotating the transportation table 116. For instance, the manipulator 130 may rotate around puppet P while the treatment device 138c is active. In this case, the treatment device 138c for sterilization and the like is one example of a treatment mechanism for performing at least one treatment among sterilization, bacteria elimination, disinfection, drying, and deodorization treatments on an object.

In addition, it is preferable that the rotating table 115, the transportation table 116, and the UV radiator be formed of an UV transmissive resin and an UV transmissive paint without causing a sense of incompatibility in terms of design. In addition, it is preferable that the treatment device 138c for sterilization and the like have a function of stopping injection of harmful light, gas, and the like when detecting an approaching human using a sensor which is not illustrated.

In addition, the robot 10 according to the first embodiment may input an object to a treatment box for sterilization and the like disposed on the support column and perform sterilization, bacteria elimination, disinfection, drying, and deodorization on the object by rotating the transportation table 116 where the object is loaded.

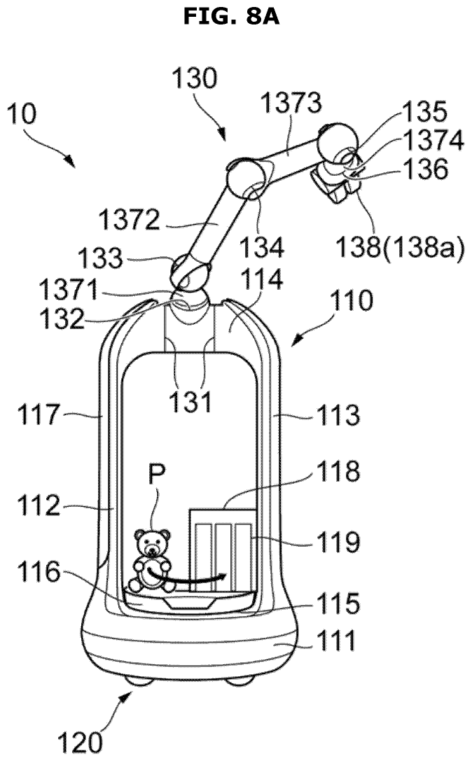

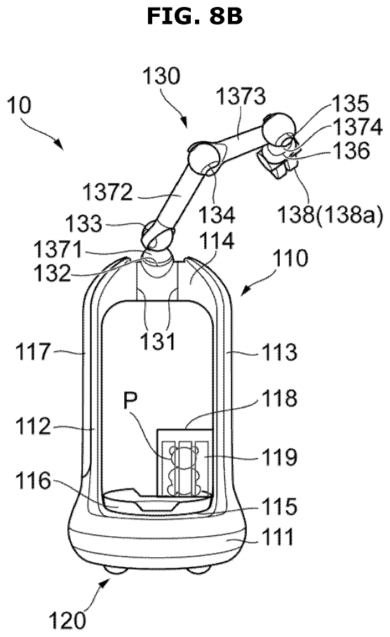

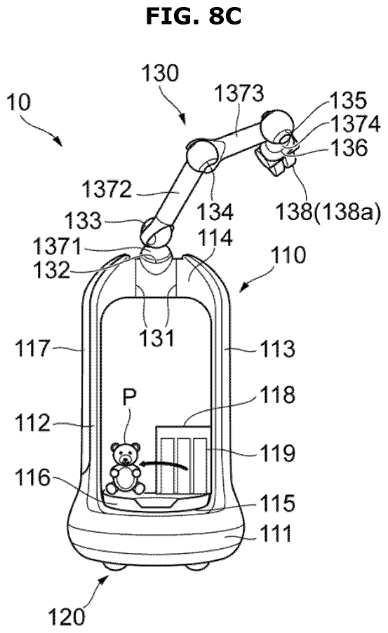

FIGS. 8A to 8C are schematic views illustrating operation of the robot 10 for the above description. In the robot 10, a treatment box 118 for sterilization and the like is disposed in the second vertical support column 113 of the main body 110. In this case, the treatment box 118 for sterilization and the like includes a leakage prevention member 119 provided at an inlet and outlet for the object to prevent a harmful substance or light ray from leaking to the outside. The leakage prevention member 119 may be, for example, a shutter or curtain.

In addition, in the robot 10, it is assumed that the puppet P which is a target object for sterilization and the like has been initially loaded at a side where the treatment box 118 for sterilization and the like of the transportation table 116 is not provided. The robot 10 may also pick the puppet P up and load the puppet P on the transportation table 116 using the manipulator 130 to implement such a state. In this state, first, the robot 10 rotates the transportation table 116 as indicated by a thick arrow in FIG. 8A. Then, as illustrated in FIG. 8B, the robot 10 may perform sterilization, bacteria elimination, disinfection, drying, and deodorization on the puppet P while the puppet P is input to the treatment box 118 for sterilization or the like. Then, the robot 10 rotates the transportation table 116 as indicated by a thick arrow in FIG. 8C to output the puppet P out of the treatment box 118 for sterilization and the like. Then, the robot 10 may lift the puppet P upward and store the puppet P at a determined position using the manipulator 130.

In this case, the treatment box 118 for sterilization or the like may be a UV radiation box, a bacterial elimination spray box, an ozone generating box, or the like. In addition, the treatment box 118 for sterilization and the like may be a box for performing at least one of sterilization, bacteria elimination, disinfection, drying, and deodorization, or a box in which functions for each performing at least one of sterilization, bacteria elimination, disinfection, drying, and deodorization are combined to perform two or more of sterilization, bacteria elimination, disinfection, drying, and deodorization. The treatment box 118 for sterilization or the like is one example of a treatment mechanism for performing at least one treatment among sterilization, bacteria elimination, disinfection, drying, and deodorization treatments when an object moves to a specific position by rotating a holder.

In addition, the robot 10 according to the first embodiment may include a cleaner unit installable on a front end of the manipulator 130.

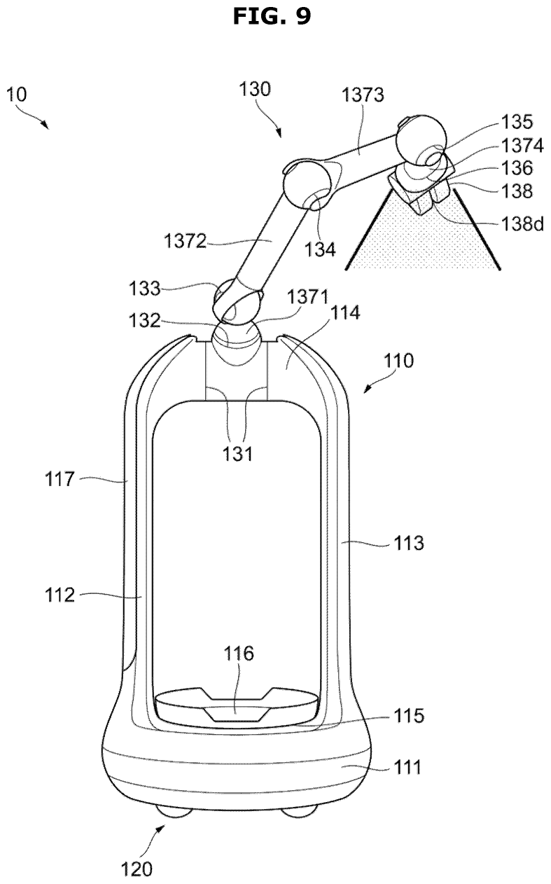

FIG. 9 is a view illustrating a configuration in which the robot 10 when the cleaner unit is installed. In the robot 10, a cleaner unit 138d is installed on the end effector 138. Accordingly, the robot 10 performs cleaning within a range surrounded by thick lines in a radial direction of a fan using the cleaner unit 138d.

In this case, the cleaner unit 138d may be provided under the main body 110 instead of the front end of the manipulator 130. Accordingly, the robot 10 can clean a floor, a wall, and a ceiling.

In addition, although not illustrated in the drawing, the cleaner unit 138d may also include a treatment device for sterilization and the like as illustrated in FIG. 7. In this case, the treatment device for sterilization may be a UV radiator, a bacteria elimination sprayer, an ozone generator, or the like. In addition, the treatment device for sterilization or the like may be a device for performing at least one of sterilization, bacteria elimination, disinfection, drying, and deodorization or a device in which devices for each performing at least one of sterilization, bacteria elimination, disinfection, drying, and deodorization are coupled to perform two or more of sterilization, bacteria elimination, disinfection, drying, and deodorization.

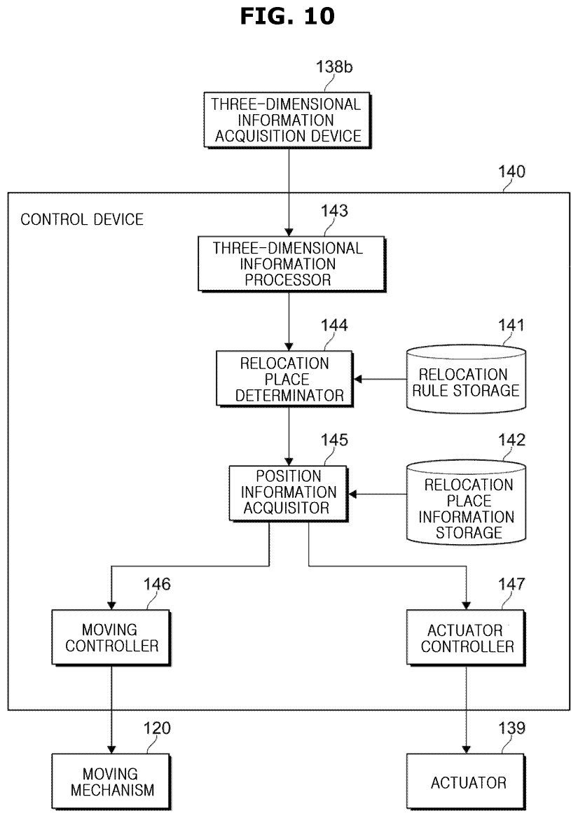

In this case, control of the robot 10 according to the first embodiment will be described. However, in this case, control of the robot 10 specifically when the robot 10 checks a three-dimensional shape of an object and performs cleaning or separating on the basis of the three-dimensional shape will be described. At this time, it is assumed that the robot 10 has loaded the object on the transportation table 116 using the manipulator 130. FIG. 10 is a view illustrating an example of a configuration of a control device 140 for performing such control of the robot 10 according to the first embodiment. The control device 140 of the robot 10 may be connected to the moving mechanism 120, the three-dimensional information acquisition device 138b disposed on the manipulator 130, and an actuator 139 for driving each joint of the manipulator 130. In addition, the control device 140 may include a relocation rule storage 141, a relocation place information storage 142, a three-dimensional information processor 143, a relocation place determinator 144, a position information acquisitor 145, a moving controller 146, and an actuator controller 147.

The relocation rule storage 141 stores a relocation rule that is a rule about where to relocate each object. For example, the relocation rule may be a rule in which types of objects and relocation places are mapped. In this case, the relocation places are places where the objects disposed on various places need to be relocated, such as shelves or boxes for storing objects, and trash cans for putting objects to be discarded.

The relocation place information storage 142 stores relocation place information indicating where the relocation places are positioned in a room. For example, the relocation place information may be information in which the relocation places and relocation place position information are mapped. In this case, the relocation place position information is provided as three-dimensional coordinates corresponding to each relocation place in a three-dimensional orthogonal coordinate system set in the room.

The three-dimensional information processor 143 obtains three-dimensional shape information of an object on the transportation table 116 from the three-dimensional information acquisition device 138b, and extracts feature information of a three-dimensional shape of the object from the three-dimensional shape information. In addition, the three-dimensional information processor 143 determines the type of object by referring to a database in which feature information of each three-dimensional shape is defined for the type of each object on the basis of the feature information of the three-dimensional shape of the object. In this case, a database independently defined by a user or a general database for recognizing an object may also be used as the database in which the feature information for the type of each object is defined.

The relocation place determinator 144 determines a relocation place by referring to the relocation rule stored in the relocation rule storage 141. The relocation rule storage 141 provides information to the relocation place determinator 144 on the basis of the type of object determined by the three-dimensional information processor 143.

The position information acquisitor 145 obtains relocation place position information by referring to the relocation place information stored in the relocation place information storage 142 on the basis of the relocation place determined by the relocation place determinator 144.

The moving controller 146 controls the moving mechanism 120 so that the robot 10 moves toward a position indicated by an X coordinate and a Y coordinate according to the relocation place position information obtained by the position information acquisitor 145. In this case, for example, position information of the robot 10 is obtained by capturing an image of a shape of the room using a camera provided in the robot 10 and interpreting the image.

The actuator controller 147 controls the actuator 139 to relocate the object to the position indicated by the X coordinate, the Y coordinate, and a Z coordinate according to the relocation place position information obtained by the position information acquisitor 145. In this case, the actuator controller 147 performs the above control after the moving controller 146 controls the moving mechanism 120 so that the robot 10 arrives at a position of the relocation place.

In addition, the control device 140 operating as described above is one example of a control mechanism for controlling a moving mechanism and a manipulator to relocate an object to a relocation place on the basis of a three-dimensional shape of the object measured by a measurement mechanism and a relationship between the three-dimensional shape and a relocation place of the object.

In this case, the control device 140 may be implemented using software and also be implemented using hardware. When the control device 140 is implemented using software, the control device 140 includes a central processing unit (CPU), a random-access memory (RAM), a read-only memory (ROM), and the like, and, for example, the CPU loads a program stored in the ROM into the RAM and executes the program. Alternatively, in a system in which an operation system (OS) is installed, software loaded into a RAM may be software stored in an external storage device such as a hard disc drive (HDD) or secure digital (SD, registered trademark) card.

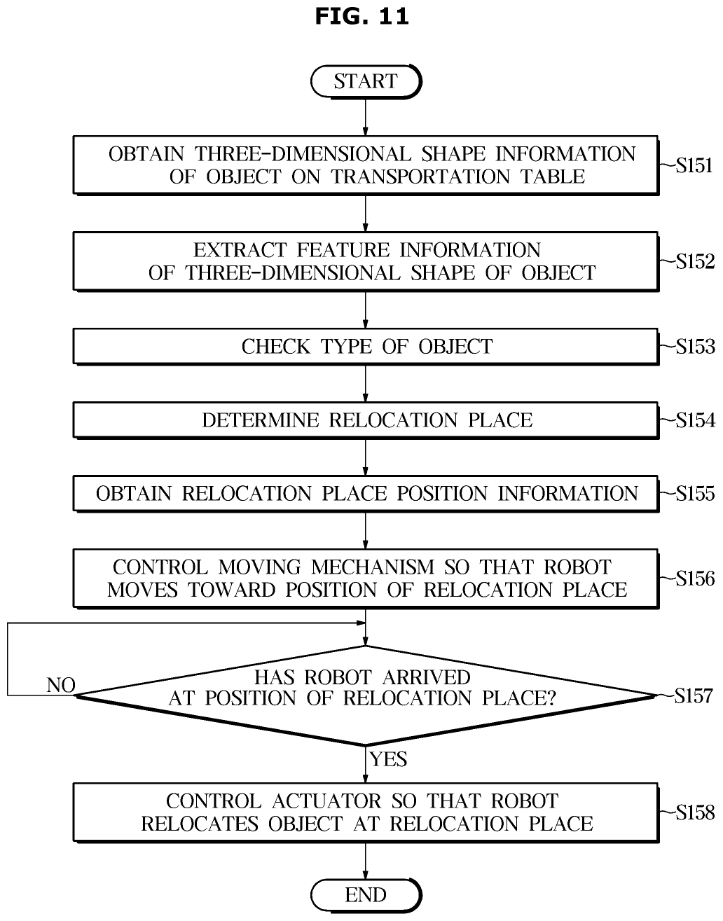

FIG. 11 is a flowchart illustrating an example of operation of the control device 140 when executing the control of the robot 10 according to the first embodiment.

In the control device 140, first, the three-dimensional information processor 143 obtains three-dimensional shape information of an object from the three-dimensional information acquisition device 138b (S151). Then, the three-dimensional information processor 143 extracts feature information of a three-dimensional shape of the object from the three-dimensional shape information of the object obtained in operation S151 (S152). In addition, the three-dimensional information processor 143 determines the type of object by referring to a database in which feature information of a three-dimensional shape for a type of each object is defined on the basis of the feature information of the three-dimensional shape of the object extracted in operation S152 (S153).

Then, in the control device 140, the relocation place determinator 144 determines a relocation place by referring to a relocation rule stored in the relocation rule storage 141 on the basis of the type of object determined in operation S153 (S154).

Then, in the control device 140, the position information acquisitor 145 obtains relocation place position information by referring to relocation place information stored in relocation place information storage 142 on the basis of the relocation place determined in operation S154 (S155).

Then, in the control device 140, the moving controller 146 controls the moving mechanism 120 so that the robot 10 moves toward a position indicated by an X coordinate and a Y coordinate for the relocation place position information obtained in operation S155 (S156).

Then, in the control device 140, the actuator controller 147 determines whether or not the robot 10 has arrived at the position indicated by the X coordinate and the Y coordinate for the relocation place position information obtained in operation S155 (S157). As a result, when it is determined that the robot 10 has not arrived at the position, the actuator controller 147 repeats operation S157. Meanwhile, when it is determined that the robot 10 has arrived at the position, the actuator controller 147 controls the actuator 139 to relocate the object at the position indicated by the X coordinate, the Y coordinate, and a Z coordinate for the relocation place position information obtained in operation S155 (S158).

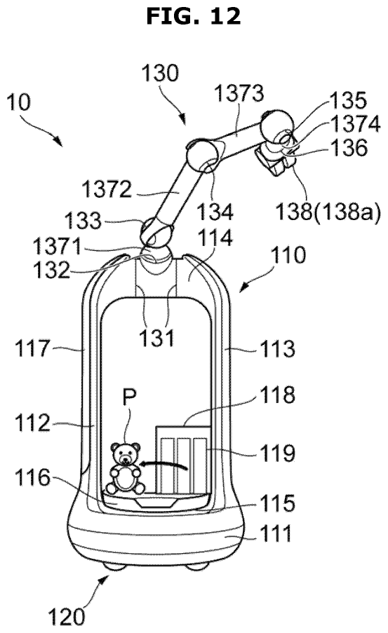

FIG. 12 is a side view illustrating a robot 20 according to a second embodiment. The robot 20 includes a main body 210, a moving mechanism 220, and a manipulator 230.

The main body 210 has a shape in which half of a door shape is removed. That is, the main body 210 includes a base 211, a vertical support column 212, and a horizontal support column 214. The base 211 is a base of the main body 210. The vertical support column 212 is a support column provided on the base 211 in a vertical direction and is one example of a first support column in the vertical direction. The horizontal support column 214 is a support column horizontally provided to be coupled to an upper end portion of the vertical support column 212 in the vertical direction and is one example of a support column in a horizontal direction.

In addition, the main body 210 includes a rotating table 215 and a transportation table 216 on the base 211. Since the rotating table 215 and the transportation table 216 are the same as the rotating table 115 and the transportation table 116 of the robot 10 according to the first embodiment, the description for the rotating table 215 and the transportation table 216 will be omitted.

In addition, in the main body 210, the vertical support column 212 includes a display 217 in a front surface of the shape in which the half of the door shape is removed. Since the display 217 is the same as the display 117 of the robot 10 according to the first embodiment, the description for the display 217 will be omitted.

The moving mechanism 220 is a mechanism for moving the main body 210. Since the moving mechanism 220 is the same as the moving mechanism 120 of the robot 10 according to the first embodiment, the description for the moving mechanism 220 will be omitted.

The manipulator 230 is a part for performing actual operation according to a use of the robot 20. The manipulator 230 is installed on the horizontal support column 214 of the main body 210. The manipulator 230 includes a first joint 231, a second joint 232, a third joint 233, a fourth joint 234, a fifth joint 235, a sixth joint 236, a first link 2371, a second link 2372, a third link 2373, a fourth link 2374, and an end effector 238.

The first joint 231 is a part corresponding to a joint installed on the horizontal support column 214 which is a root of the manipulator 230 and is installed to be rotated 360° or more about the horizontal support column 214 as a rotation axis. Accordingly, the manipulator 230 may lift the object upward.

Since the other components of the manipulator 230 are the same as other components of the manipulator 130 of the robot 10 according to the first embodiment, the description for the other components of the manipulator 230 will be omitted. That is, a rotation direction of each joint of the manipulator 230 is the same as that illustrated in FIG. 5.

Since there is no support column in dotted line A in the robot 20 according to the second embodiment, rigidity decreases, but an operating range of the manipulator 230 increases.

In addition, the operation illustrated in FIGS. 6 to 8, the configuration illustrated in FIG. 9, and the configuration and the operation of the control device illustrated in FIGS. 10 and 11 are also the same as those of the robot 20 according to the second embodiment.

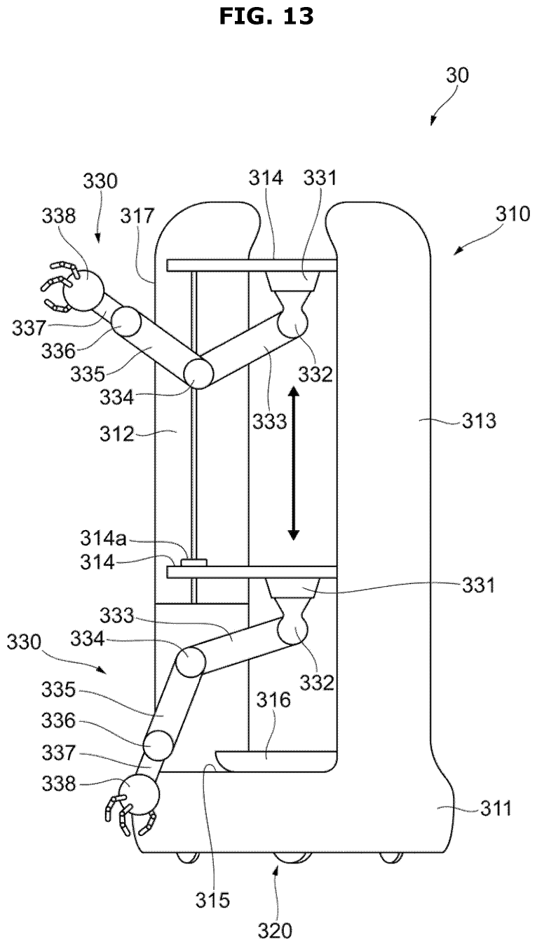

FIG. 13 is a side view illustrating a robot 30 according to a third embodiment. The robot 30 includes a main body 310, a moving mechanism 320, and a manipulator 330.

The main body 310 includes a base 311, a first vertical support column 312, a second vertical support column 313, and a horizontal support column 314. The base 311 is a base of the main body 310. The first vertical support column 312 is a first support column provided on the base 311 in a vertical direction and one example of a first support column in the vertical direction. The second vertical support column 313 is a second support column provided on the base 311 in a vertical direction and is one example of a second support column in the vertical direction. The horizontal support column 314 is a support column provided in a horizontal direction to be moved in the vertical direction as indicated by a thick arrow between the first vertical support column 312 and the second vertical support column 313 and is one example of a support column in the horizontal direction. In this case, the horizontal support column 314 may be configured to move in the vertical direction using a screw 314a. The screw may be, for example, a ball screw or transfer screw.

In addition, the main body 310 includes a rotating table 315 and a transportation table 316 on the base 311. Since the rotating table 315 and the transportation table 316 are the same as the rotating table 115 and transportation table 116 of the robot 10 according to the first embodiment, the description for the rotating table 315 and the transportation table 316 will be omitted.

In addition, in the main body 310, the first vertical support column 312 includes a display 317. Since the display 317 is the same as the display 117 of the robot 10 according to the first embodiment, the description for the display 317 will be omitted.

The moving mechanism 320 is a mechanism for moving the main body 310. Since the moving mechanism 320 is the same as the moving mechanism 120 of the robot 10 according to the first embodiment, the description for the moving mechanism 320 will be omitted.

The manipulator 330 is a part for performing actual operation according to a use of the robot 30. The manipulator 330 is installed on the horizontal support column 314 of the main body 310. The manipulator 330 includes a first joint 331, a second joint 332, a first link 333, a third joint 334, a second link 335, a fourth joint 336, a wrist 337, and an end effector 338.

The first joint 331 is a part corresponding to a joint which is a root of the manipulator 330 and is installed on the horizontal support column 314 of the main body 310 to face downward. In this case, the first joint 331 may be installed to be rotated 360° or more about the horizontal support column 314 as a rotation axis.

Since other components of the manipulator 330 are the same as other components of the manipulator 130 of the robot 10 according to the first embodiment, the description for the other components of the manipulator 330 will be omitted. That is, each joint, each link, the wrist 337, and a rotation direction of the end effector 338 of the manipulator 330 are the same as those illustrated in FIG. 5.

In addition, the operation illustrated in FIGS. 6 to 8, the configuration illustrated in FIG. 9, and the configuration and the operation of the control device illustrated in FIGS. 10 and 11 are the same as those in the robot 30 according to the third embodiment.

In addition, in the third embodiment, when the main body 310 has the same door shape as in the first embodiment, the horizontal support column 314 is movable in the vertical direction but is not limited thereto. When the main body 310 has a shape in which half of a door shape is removed like the second embodiment, the horizontal support column 314 may also be movable in the vertical direction.

In the robot according to the first to third embodiments, although the main body includes one or two vertical support columns and the horizontal support column connected to vertical support columns, and the manipulator is connected to the horizontal support column, but the present disclosure is not limited thereto. The main body includes at least one support column, one of the support columns is a horizontal support column, and a manipulator is connected to the support column in a horizontal direction.

In addition, a main body may also include at least one support column, one of the support columns may be a vertical support column, and a manipulator may also be connected to the vertical support column.

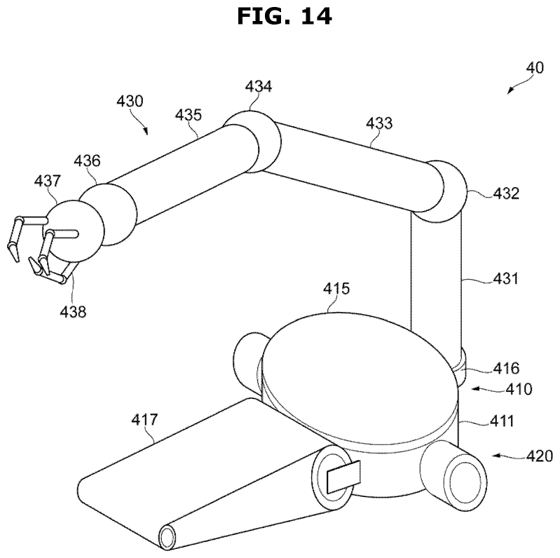

FIG. 14 is a top perspective view illustrating a robot 40 according to a fourth embodiment. The robot 40 includes a main body 410, a moving mechanism 420, and a manipulator 430.

The main body 410 includes a base 411, a rotating table 415, a support 416, and a carry-in mechanism 417. The base 411 is a base of the main body 410. The rotating table 415 is a flat table rotated within a horizontal surface provided on the base 411 and is one example of a holder rotated within a horizontal plane for loading an object. The support 416 is a part for supporting the manipulator 430 provided at a position tangent to an outer circumference of the rotating table 415. The carry-in mechanism 417 is a member for connecting a floor and an upper surface of the rotating table 415 through a gentle slope so that the manipulator 430 may smoothly load an object on the rotating table 415.

The moving mechanism 420 is a mechanism for moving the main body 410. In the drawing, the moving mechanism 420 is implemented by arranging a tire beside the main body 410 but may also be implemented by arranging a crawler under the main body 410.

The manipulator 430 is a part for performing actual operation according to a use of the robot 40. The manipulator 430 is installed on the support 416 of the main body 410. The manipulator 430 includes a first link 431, a first joint 432, a second link 433, a second joint 434, a third link 435, a third joint 436, a wrist 437, and an end effector 438. The manipulator 430 is capable of rotating 360 degrees or more about a central axis of support 416.

The first link 431 is a part corresponding to an arm installed on the support 416. The first joint 432 is a part corresponding to a joint of a front end of the first link 431. The second link 433 is a part corresponding to an arm installed on the first joint 432. The second joint 434 is a part corresponding to a joint of a front end of the second link 433. The third link 435 is a part corresponding to an arm installed on the second joint 434. The third joint 436 is a part corresponding to a joint of a front end of the third link 435.

The wrist 437 is a part corresponding to a wrist installed on the third joint 436. The end effector 438 is a device installed on the wrist 437 to perform the actual operation according to the use of the robot 40.

In this case, the operation illustrated in FIGS. 6 to 8, the configuration illustrated in FIG. 9, and the configuration and the operation of the control device illustrated in FIGS. 10 and 11 are the same as those in the robot 40 according to the fourth embodiment.

As described above, in present embodiments, a robot is formed by forming a main body having a door shape on a moving mechanism and arranging a manipulator on the main body. Accordingly, a large operating range of the manipulator of the robot can be secured. As a result, for example, the robot is no need to move while repeatedly approaching and lifting objects upward in order to transport objects distributed in a wide area.

In addition, a transportation table on which an object is loaded and which is rotated is provided on a robot. Accordingly, the robot can be applied to various uses such as object transportation, measurement for a three-dimensional shape of the object, sterilization and disinfection for the object, and the like.

What is claimed is:

1. A robot comprising:
a main body comprising at least one support column;
a moving mechanism configured to move the main body; and
a manipulator installed on one support column of the at least one support column,
wherein the manipulator can be rotated 360° or more about a rotation axis,
wherein the rotation axis corresponds to an axis of the one support column.

2. The robot of claim 1, wherein:
the at least one support column comprises a support column in a horizontal direction; and
the one support column is the support column in the horizontal direction.

3. The robot of claim 2, wherein:
the at least one support column further comprises a first support column in a vertical direction; and
a first end portion of the support column in the horizontal direction is coupled to an upper end portion of the first support column in the vertical direction.

4. The robot of claim 3, wherein:
the at least one support column further comprises a second support column in the vertical direction; and
a second end portion of the support column in the horizontal direction is coupled to an upper end portion of the second support column in the vertical direction.

5. The robot of claim 2, wherein:
the at least one support column further comprises a first support column in a vertical direction; and
a first end portion of the support column in the horizontal direction is disposed on the first support column to be moved in the vertical direction.

6. The robot of claim 5, wherein:
the at least one support column further comprises a second support column in the vertical direction; and
a second end portion of the support column in the horizontal direction is disposed on the second support column to be moved in the vertical direction.

7. The robot of claim 1, wherein:
the at least one support column comprises a support column in a vertical direction; and
the one support column is the support column in the vertical direction.

8. The robot of claim 1, further comprising a measurement mechanism configured to measure a three-dimensional shape of an object.

9. The robot of claim 1, further comprising a treatment mechanism configured to perform at least one treatment among sterilization, bacteria elimination, disinfection, drying, and deodorization treatments on an object.

10. The robot of claim 1, wherein the main body further comprises a holder rotated within a horizontal plane to hold an object.

11. The robot of claim 10, further comprising a measurement mechanism configured to measure a three-dimensional shape of a corresponding object by rotating the holder holding the object.

12. The robot of claim 11, further comprising a controller configured to control the moving mechanism and the manipulator to relocate the corresponding object to a corresponding relocation position on the basis of the three-dimensional shape of the object measured by the measurement mechanism and a relationship between the corresponding three-dimensional shape and the relocation position of the corresponding object.

13. The robot of claim 10, further comprising a treatment mechanism configured to perform at least one treatment among sterilization, bacteria elimination, disinfection, drying, removal, and deodorization treatments on the corresponding object by rotating the holder holding the object.

14. The robot of claim 13, wherein the treatment mechanism performs the at least one treatment on the object moved at a specific position by rotating the holder.

15. The robot of claim 1, further comprising a cleaner unit disposed on the manipulator.
